(19) United States
(12) Patent Application Publication
(10) Pub. No.: US 2024/0108502 A1
(43) Pub. Date: Apr. 4, 2024

(54) DEVICE TO PROTECT ENDOTHELIUM DURING OPHTHALMIC SURGERY

(71) Applicant: EyeYon Medical Ltd., Nes Ziona (IL)

(72) Inventors: Ofer Daphna, Beit Elazari (IL); Nahum Ferera, Beit Elazari (IL); Dmitry Dubson, Rehovot (IL)

(73) Assignee: EyeYon Medical Ltd., Nes Ziona (IL)

(21) Appl. No.: 17/937,504

(22) Filed: Oct. 3, 2022

(51) Int. Cl. A61F 9/007

(52) U.S. Cl. CPC A61F 9/007

(57) ABSTRACT

An ocular implant includes an endothelial protective implant which is dome-shaped and constructed of a clear, transparent, biologically compatible material, and a grasping member that extends outwards from a periphery of the endothelial protective implant.

### FIELD OF THE INVENTION

The present invention relates generally to endothelial implants, and particularly to an endothelial protective implant for protecting the endothelium during different ophthalmic procedures.

### BACKGROUND OF THE INVENTION

Known complications that can arise after cataract surgery include pseudophakic bullous keratopathy (PBK) and aphakic bullous keratopathy (ABK), both of which involve development of irreversible corneal edema. As corneal edema progresses and worsens, first stromal and then intercellular epithelial edema develops. Epithelial edema is associated with the development of bullae; hence, the name bullous keratopathy

In addition, the mechanical process of cataract extraction can create endothelial damage that may result in irreversible corneal edema.

To protect the cornea, the prior art applies gel on the posterior portion of the cornea at the beginning of surgery. The gel is supposed to protect the cornea but is not always effective.

### SUMMARY

The present invention relates to an endothelial protective implant for protecting the endothelium, as is described more in detail hereinbelow.

The endothelial protective implant is a mechanical shield that may be introduced at the beginning of the surgery. The shield may be attached to the posterior part of the cornea at the beginning of surgery with viscoelastic gel for adhesion, and removed at the end of surgery.

There is provided in accordance with a non-limiting embodiment of the present invention an ocular implant including an endothelial protective implant which is dome-shaped and constructed of a clear, transparent, biologically compatible material, and a grasping member that extends outwards from a periphery of the endothelial protective implant.

The grasping member may be straight or curved.

There is provided in accordance with a non-limiting embodiment of the present invention a method for protecting an endothelium during an ophthalmic procedure, including providing an endothelial protective implant which is dome-shaped and constructed of a clear, transparent, biologically compatible material, instructing to attach the endothelial protective implant to a posterior portion of a cornea of an eye, instructing to perform an ophthalmic procedure while the endothelial protective implant remains attached to the cornea, and instructing to remove the endothelial protective implant from the eye.

In accordance with a non-limiting embodiment of the present invention instructing to attach the endothelial protective implant to the posterior portion of the cornea of the eye includes instructing to use a viscoelastic gel to adhere the endothelial protective implant to the posterior portion of the cornea of the eye.

In accordance with a non-limiting embodiment of the present invention a grasping member extends outwards from a periphery of the endothelial protective implant, and the method further includes instructing to grasp the grasping member to remove the endothelial protective implant from the eye.

### BRIEF DESCRIPTION OF THE DRAWINGS

The present invention will be understood and appreciated more fully from the following detailed description taken in conjunction with the drawings in which:

FIG. 1 is a simplified perspective illustration of an endothelial protective implant, constructed and operative in accordance with a non-limiting embodiment of the present invention; and

FIG. 2 is a simplified illustration of the endothelial protective implant implanted on a posterior portion of the cornea, in accordance with a non-limiting embodiment of the present invention.

### DETAILED DESCRIPTION

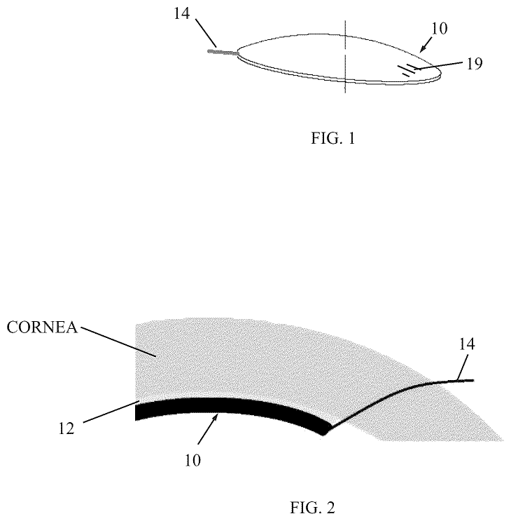

Reference is now made to FIG. 1, which illustrates an endothelial protective implant 10, constructed and operative in accordance with a non-limiting embodiment of the present invention.

Implant 10 may be dome-shaped and constructed of a clear, transparent, biologically compatible material, such as but not limited to, polymethylmethacrylate (PMMA), silicone, silicone rubber, collagen, hyaluronic acid (including the sodium, potassium and other salts thereof), hydrogel, such as acrylic or methacrylic hydrogels, e.g., hydroxyethyl methacrylate or methacrylic acid copolymer/partially hydrolyzed poly(2-hydroxyethyl methacrylate) (known as PolyHEMA), polysulfones, thermolabile materials and other relatively hard or relatively soft and flexible biologically inert optical materials, or any combination of such materials, such as a gel encapsulated in a polymer. Implant 10 may thus be rigid, semi-rigid or foldable, for example. Some or all of implant 10 may be hydrophilic or hydrophobic.

Implant 10 may be made of a copolymer of hydroxyethyl methacrylate and methyl methacrylate, commercially available as Ci26 from Contamac Ltd., Saffron Walden, Essex, UK. Ci26 is a random, crosslinked, acrylate based copolymer consisting of poly [(methylmethacrylate)-co-(2-hydroxyethyl methacrylate)-co-(ethylene glycol dimethacrylate)], that is, it is a copolymer of methylmethacrylate (MMA) and 2-hydroxyethyl methacrylate (HEMA) and ethylene glycol dimethacrylate (EGDM). Methyl methacrylate (MMA) is a hydrophobic monomer that forms a homopolymer that does not substantially absorb water. 2-hydroxyethyl methacrylate (HEMA) is a modification of MMA, in which the non-polar pendant methyl group of MMA is replaced with a polar hydroxyethyl functional group. When HEMA is made into a homopolymer (pHEMA), it retains a hydrophobic backbone structure but the polar pendant groups allow water to be absorbed into the polymer matrix. Fully hydrated hydrogels of pHEMA typically contain up to 40% water by weight. EGDM contains two methacrylate functionalities polymerized to form cross-links between the polymer chains, MMA and HEMA, and is hydrophobic in nature. Ci26 is a blend of approximately 14% MMA, 85% HEMA, and <1% of EGDM, producing a material that can absorb water, and when fully hydrated will contain 26% water by weight. The material therefore contains a mixture of both hydrophilic and hydrophobic components.

The average radius of the posterior corneal surface has been measured as 6.53±0.25 mm. Accordingly, implant 10 may have a radius of 6.53±0.25 mm or a radius in the range of 4-6 mm, or in the range of 3-6.5 mm, or in the range of 3-5 mm. The implant 10 may be constructed as a 50 microns lens, or 40-50 microns lens, or 30-50 microns lens, or 20-40 microns lens.

The radius of curvature of implant 10 may be virtually the same as the radius of curvature of the posterior corneal surface. The radius of curvature of implant 10 may be 6-8 mm, or at least 7 mm, or between 7-12 mm, or between 10-12 mm.

Implant 10 may be inserted into the eye, without limitation, through a 1.8-3.0 mm incision, alternatively a 1.8-2.7 mm incision, alternatively a 1.8-2.4 mm incision, alternatively a 2.0-2.4 mm incision, and preferably a 2.2-2.4 mm incision.

Implant 10 may be attached to the posterior portion of the cornea at the beginning of surgery with a viscoelastic gel 12 for adhesion, and the implant 10 may be removed at the end of surgery.

Implant 10 may include a grasping member 14 that extends outwards from a periphery of implant 10. Grasping member 14 may facilitate removal of implant 10 by grasping it with any appropriate tool or by hand. Grasping member 14 may be made of the same or of a different material than implant 10. Grasping member 14 may be straight as shown in FIG. 1, or curved as shown in FIG. 2.

In accordance with a non-limiting embodiment of the invention, implant 10 may include a drug-coating or drug-eluting patch 19 that can release an anti-fungal drug, an antiviral drug, or an antibacterial drug.

What is claimed is:

1. An ocular implant comprising:
an endothelial protective implant which is dome-shaped and constructed of a clear, transparent, biologically compatible material, and a grasping member that extends outwards from a periphery of said endothelial protective implant.

2. The ocular implant according to claim 1, wherein said grasping member is straight.

3. The ocular implant according to claim 1, wherein said grasping member is curved.

4. The ocular implant according to claim 1, wherein said endothelial protective implant comprises a drug-coating or a drug-eluting patch.

5. A method for protecting an endothelium during an ophthalmic procedure, comprising:
providing an endothelial protective implant which is dome-shaped and constructed of a clear, transparent, biologically compatible material;
instructing to attach said endothelial protective implant to a posterior portion of a cornea of an eye;
instructing to perform an ophthalmic procedure while said endothelial protective implant remains attached to the cornea; and
instructing to remove said endothelial protective implant from the eye.

6. The method according to claim 5, wherein instructing to attach said endothelial protective implant to the posterior portion of the cornea of the eye comprises instructing to use a viscoelastic gel to adhere said endothelial protective implant to the posterior portion of the cornea of the eye.

7. The method according to claim 6, wherein a grasping member extends outwards from a periphery of said endothelial protective implant, and further comprising instructing to grasp said grasping member to remove said endothelial protective implant from the eye.
